(19) United States
(12) Patent Application Publication
(10) Pub. No.: US 2023/0255634 A1
(43) Pub. Date: Aug. 17, 2023

(54) IMPLANTABLE MEDICAL DEVICE WITH OPPOSING BARBS FOR BI-DIRECTIONAL STABILITY

(71) Applicant: BOSTON SCIENTIFIC SCIMED, INC., MAPLE GROVE, MN (US)

(72) Inventors: Joshua Mark Inouye, Brooklyn Park, MN (US); James M. Anderson, Corcoran, MN (US)

(73) Assignee: BOSTON SCIENTIFIC SCIMED, INC., MAPLE GROVE, MN (US)

(21) Appl. No.: 18/108,099

(22) Filed: Feb. 10, 2023

Related U.S. Application Data
(60) Provisional application No. 63/309,105, filed on Feb. 11, 2022.

(51) Int. Cl. A61B 17/12

(52) U.S. Cl. CPC A61B 17/12122; A61B 17/12172

(57) ABSTRACT

An implantable medical device includes an expandable frame that is moveable between a collapsed configuration for delivery and an expanded configuration for deployment. One or more proximally-facing fixation burrs are secured relative to the expandable frame. One or more distally-facing fixation burrs are secured relative to the expandable frame. The one or more proximally-facing fixation burrs and the one or more distally-facing fixation burrs together provide the implantable medical device with bi-directional stability.

### CROSS-REFERENCE TO RELATED APPLICATIONS

This application claims the benefit of priority of U.S. Provisional Application No. 63/309,105 filed Feb. 11, 2022, the entire disclosure of which is hereby incorporated by reference.

### TECHNICAL FIELD

The present disclosure pertains to medical devices, and methods for manufacturing and using medical devices. More particularly, the disclosure is directed to implantable medical devices having opposing barbs for bi-directional stability.

### BACKGROUND

A wide variety of medical devices have been developed for medical use, for example, for use in accessing body cavities and interacting with fluids and structures in body cavities. Some of these devices may include guidewires, catheters, pumps, motors, controllers, filters, grinders, needles, valves, and delivery devices and/or systems used for delivering such devices. These devices are manufactured by any one of a variety of different manufacturing methods and may be used according to any one of a variety of methods. Of the known medical devices and methods, each has certain advantages and disadvantages.

### SUMMARY

This disclosure provides design, material, manufacturing method, and use alternatives for medical devices. As an example, an implantable medical device includes an expandable frame defining a body of the implantable medical device, the expandable frame moveable between a collapsed configuration for delivery and an expanded configuration for deployment. One or more proximally-facing fixation burrs are secured relative to the expandable frame. One or more distally-facing fixation burrs are secured relative to the expandable frame. The one or more proximally-facing fixation burrs and the one or more distally-facing fixation burrs together are adapted to provide the implantable medical device with bi-directional stability.

Alternatively or additionally, the one or more proximally-facing fixation burrs may be disposed within a distal region of the expandable frame.

Alternatively or additionally, the one or more distally-facing fixation burrs may be disposed within a proximal region of the expandable frame.

Alternatively or additionally, the expandable frame may include a plurality of struts.

Alternatively or additionally, the one or more distally-facing fixation burrs may be secured to at least some of the plurality of struts.

Alternatively or additionally, the one or more proximally-facing fixation burrs may be secured to at least some of the plurality of struts.

Alternatively or additionally, the implantable medical device may include an LAAC (left atrial appendage closure) device.

Alternatively or additionally, the implantable medical device may include a heart valve implant.

Alternatively or additionally, the expandable frame may be biased to the expanded configuration.

As another example, a left atrial appendage closure device includes an expandable frame that is moveable between a collapsed configuration for delivery and an expanded configuration for deployment. One or more proximally-facing fixation burrs are secured relative to the expandable frame. One or more distally-facing fixation burrs are secured relative to the expandable frame. The one or more proximally-facing fixation burrs and the one or more distally-facing fixation burrs together are adapted to provide the left atrial appendage closure device with bi-directional stability.

Alternatively or additionally, the one or more proximally-facing fixation burrs may be disposed within a distal region of the expandable frame.

Alternatively or additionally, the one or more distally-facing fixation burrs may be disposed within a proximal region of the expandable frame.

Alternatively or additionally, the expandable frame may be adapted to enable the expandable frame to be partially recaptured within a delivery device for repositioning the expandable frame, wherein the distally-facing fixation burrs may be disposed within the delivery device when the expandable frame is partially recaptured.

Alternatively or additionally, the expandable frame may be biased to the expanded configuration.

Alternatively or additionally, the expandable frame may include a shape memory material.

Alternatively or additionally, the expandable frame may include a plurality of laser-cut struts, and the proximally-facing fixation struts may be laser cut as part of the laser-cut struts.

Alternatively or additionally, the distally-facing fixation struts may be welded to the laser-cut struts.

Alternatively or additionally, wherein the left atrial appendage closure device further includes a membrane covering at least a proximal portion of the expandable frame.

As another example, an atrial appendage closure device, includes an expandable braided frame that is moveable between a collapsed configuration for delivery and an expanded configuration for deployment. One or more proximally-facing fixation burrs are secured to the expandable braided frame and one or more distally-facing fixation burrs are secured to the expandable braided frame. The one or more proximally-facing fixation burrs and the one or more distally-facing fixation burrs together are adapted to provide the left atrial appendage closure device with bi-directional stability.

Alternatively or additionally, the one or more proximally-facing fixation burrs and/or the one or more distally-facing fixation burrs may include wire elements that are separately formed and subsequently attached to the expandable braided frame.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a schematic side view of an illustrative implantable medical device;

FIG. 2 shows the illustrative implantable medical device of FIG. 1 partially recaptured;

FIG. 3 is a side view of an illustrative left atrial appendage closure device;

FIG. 4 is a side view of an illustrative left atrial appendage closure device including a fabric covering;

FIG. 5 is a schematic side view of an illustrative implantable medical device;

FIG. 6 is a side view of an illustrative aortic valve implant frame; and

FIG. 7 is a side view of an illustrative aortic valve implant frame.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

A variety of different implantable medical devices are implanted within patients. In some instances, implantable medical devices may be subject to movement after implantation, and thus some include structure such as fixation burrs that assist in securing the implantable medical device in place. In some cases, some implantable medical devices can still move in place, which can cause embolization. Generally, prior art devices have included fixation burrs that only extend in a single direction. Having burrs that extend only in a single direction can be beneficial when needing to recapture and reposition an implantable medical device that includes burrs.

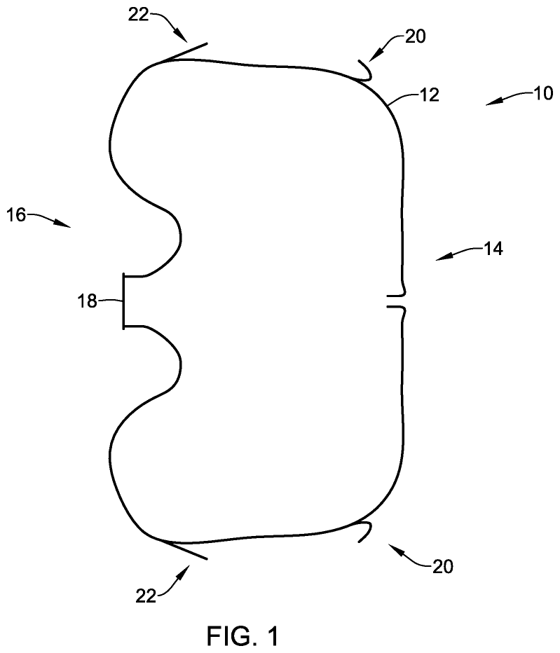

FIG. 1 is a schematic side view of an illustrative implantable medical device (IMD) 10 that is adapted to have bi-directional stability. As a result, the IMD 10 may have relatively fewer issues with movement-caused embolization. The implantable medical device 10 includes an expandable frame 12. For clarity, only the outer extent of the expandable frame 12 is shown. It will be appreciated that the expandable frame 12 may include one or more, or even a plurality, of internal struts or other members that fill at least some of the space within the expandable frame 12 as shown.

The expandable frame 12 may be moveable between a collapsed configuration for delivery and an expanded configuration for deployment. In FIG. 1, the expandable frame 12 is shown in the expanded configuration. In some cases, the expandable frame 12 may be biased to the expanded configuration, and is adapted to permit collapse of the expandable frame 12 in response to a compressive force applied to the expandable frame 12. For example, pulling the expandable frame 12 into a delivery device may be construed as collapsing the expandable frame 12 into the collapsed configuration. Moving the expandable frame 12 out of the delivery device allows the expandable frame 12 to naturally regain the expanded configuration to which it is biased.

The IMD 10, which in some cases may be considered as representing an LAAC (left atrial appendage closure) device, may be considered as having a distal region 14 and a proximal region 16. In this, distal and proximal refer to the anticipated orientation of the IMD 10 once the IMD 10 has been successfully implanted.

The IMD 10 includes a hub 18 that is disposed within the proximal region 16 of the IMD 10. The hub 18 may be used for releasably securing the IMD 10 to a delivery device (not shown), for example. In some cases, the hub 18 may serve as an attachment point for some of the internal components of the expandable frame 12 that are not shown in this schematic view.

The IMD 10 includes one or more proximally-facing fixation barbs 20. While only two proximally-facing fixation barbs 20 are shown, it will be appreciated that the IMD 10 may include any number of proximally-facing fixation barbs 20, and may include three, four, five, six, seven, eight, nine, ten or more proximally-facing fixation barbs 20. The proximally-facing fixation barbs 20 may be integrally formed with the expandable frame 12, particularly if for example the expandable frame 12 is laser-cut. In some cases, the proximally-facing fixation barbs 20 may be formed of wire and subsequently secured to the expandable frame 12 using any desired technique such as but not limited to welding or soldering. The proximally-facing fixation barbs 20 may be considered as being proximally-facing when the IMD 10 has been implanted.

The IMD 10 includes one or more distally-facing fixation barbs 22. While only two distally-facing fixation barbs 22 are shown, it will be appreciated that the IMD 10 may include any number of distally-facing fixation barbs 22, and may include three, four, five, six, seven, eight, nine, ten or more distally-facing fixation barbs 20. The distally-facing fixation barbs 20 may be integrally formed with the expandable frame 12, particularly if for example the expandable frame 12 is laser-cut. In some cases, the distally-facing fixation barbs 20 may be formed of wire and subsequently secured to the expandable frame 12 using any desired technique such as but not limited to welding or soldering. The distally-facing fixation barbs 22 may be considered as being distally-facing when the IMD 10 has been implanted.

The proximally-facing fixation barbs 20 and the distally-facing fixation barbs 22 may be disposed anywhere on the expandable frame 12. In some instances, as shown, the proximally-facing fixation barbs 20 are located within the distal region 14 and the distally-facing fixation barbs 22 are located within the proximal region 16. In some cases, the proximally-facing fixation barbs 20 are located within the proximal region 16 and the distally-facing fixation barbs 22 are located within the distal region 14. In some instances, the proximally-facing fixation barbs 20 and the distally-facing fixation barbs 22 may be randomly distributed throughout the expandable frame 12.

While the proximally-facing fixation barbs 20 and the distally-facing fixation barbs 22 are shown and described as being either proximally-facing or distally-facing, it will be appreciated that in some instances, the fixation barbs may not necessarily be proximally-facing or distally-facing. In some cases, the fixation barbs may be considered to be divided into a first group of one or more fixation barbs that extend in a first direction, and a second group of one or more fixation barbs that extend in a second direction that oppose the first direction. The first group of fixation barbs may extend in a direction that is 180 degrees apart from the direction in which the second group of fixation barbs extend. The first group of fixation barbs may extend in a direction that is 170 degrees apart, or 160 degrees apart, or 150 degrees apart, or 140 degrees apart, or 130 degrees apart, or 120 degrees apart, or even less.

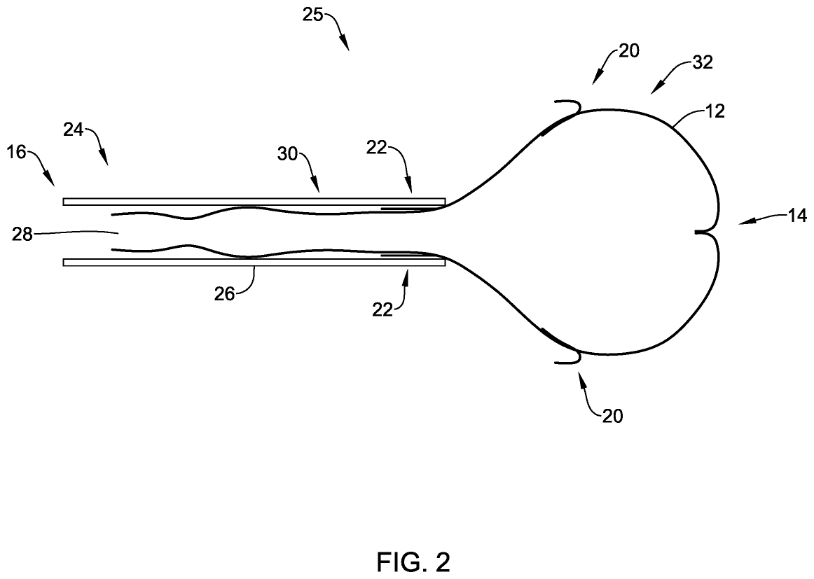

In some instances, there may be a desire to at least partially recapture and subsequently reposition the IMD 10. FIG. 2 shows an assembly 25 that includes the IMD 10 partially recaptured within a delivery device 24. The delivery device 24, which is shown schematically, includes a sheath 26 that is adapted to accommodate the IMD 10 within a lumen 28 defined by the sheath 26. As seen in FIG. 2, a portion 30 of the expandable frame 12 has been pulled proximally into the lumen 28, with a portion 32 of the expandable frame 12 remaining outside of the lumen 28.

It will be appreciated that the assembly 25 may be advanced distally through a patient's vasculature with the implantable medical device 10 partially recaptured within the delivery device 24, as shown. The proximally-facing fixation barbs 20 (which as noted are proximally-facing when the implantable medical device 10 has been implanted) remain outside of the lumen 28, but since they are proximally-facing, the proximally-facing fixation barbs 20 are not an issue with advancing the assembly 25 distally. The distally-facing fixation barbs 22 (which are noted are distally-facing when the IMD 10 has been implanted) have been pulled within the lumen 28 and thus are not an issue with advancing the assembly 25 distally. In some cases, particularly if there are distally-facing fixation barbs located within the distal region 14, it may be beneficial to completely recapture the IMD 10 within the delivery device 24 prior to advancing the assembly 25 distally to reach an alternate or adjusted deployment site, for example.

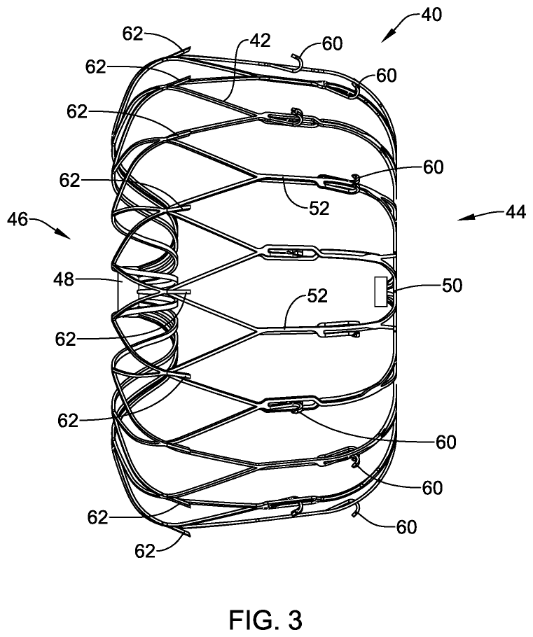

FIG. 3 is a side view of an illustrative left atrial appendage closure (LAAC) device 40 that may be considered as being an example of the IMD 10. The LAAC device 40 includes an expandable frame 42. The expandable frame 42 may be moveable between a collapsed configuration for delivery and an expanded configuration for deployment.

In FIG. 3, the expandable frame 32 is shown in the expanded configuration. In some cases, the expandable frame 32 may be biased to the expanded configuration, and is adapted to permit collapse of the expandable frame 32 in response to a compressive force applied to the expandable frame 32. For example, pulling the expandable frame 32 into a delivery device may be construed as collapsing the expandable frame 32 into the collapsed configuration. Moving the expandable frame 32 out of the delivery device allows the expandable frame 32 to naturally regain the expanded configuration to which it is biased.

The LAAC device 40 may be considered as having a distal region 44 and a proximal region 46. In this, distal and proximal refer to the anticipated orientation of the LAAC 40 once the LAAC 40 has been successfully implanted.

The LAAC device 40 includes a hub 48 that is disposed within the proximal region 46 of the LAAC device 40. The hub 48 may be used for releasably securing the LAAC device 40 to a delivery device (not shown), for example. In some cases, the hub 48 may serve as an attachment point for some of the internal components of the expandable frame 42. The LAAC device 40 may also include a secondary hub 50 that is disposed within the distal region 44 of the expandable frame 42. The expandable frame 42 includes a number of struts 52 that extend from the hub 48 to the secondary hub 50.

The LAAC device 40 includes a number of proximally-facing fixation barbs 60. The proximally-facing fixation barbs 60 may be integrally formed with the expandable frame 42, particularly if, as shown, the expandable frame 42 is laser-cut. In some cases, the proximally-facing fixation barbs 60 may be formed of wire and subsequently secured to the expandable frame 42 using any desired technique such as but not limited to welding or soldering. The proximally-facing fixation barbs 60 may be considered as being proximally-facing when the LAAC device 40 has been implanted.

The LAAC device 40 includes a number of distally-facing fixation barbs 62. The distally-facing fixation barbs 62 may be integrally formed with the expandable frame 42, particularly if for example the expandable frame 42 is laser-cut. In some cases, the distally-facing fixation barbs 62 may be formed of wire and subsequently secured to the expandable frame 42 using any desired technique such as but not limited to welding or soldering. The distally-facing fixation barbs 62 may be considered as being distally-facing when the LAAC device 40 has been implanted.

While the proximally-facing fixation barbs 60 and the distally-facing fixation barbs 62 are shown and described as being either proximally-facing or distally-facing, it will be appreciated that in some instances, the fixation barbs may not necessarily be proximally-facing or distally-facing. In some cases, the fixation barbs may be considered to be divided into a first group of one or more fixation barbs that extend in a first direction, and a second group of one or more fixation barbs that extend in a second direction that oppose the first direction. The first group of fixation barbs may extend in a direction that is 180 degrees apart from the direction in which the second group of fixation barbs extend. The first group of fixation barbs may extend in a direction that is 170 degrees apart, or 160 degrees apart, or 150 degrees apart, or 140 degrees apart, or 130 degrees apart, or 120 degrees apart, or even less.

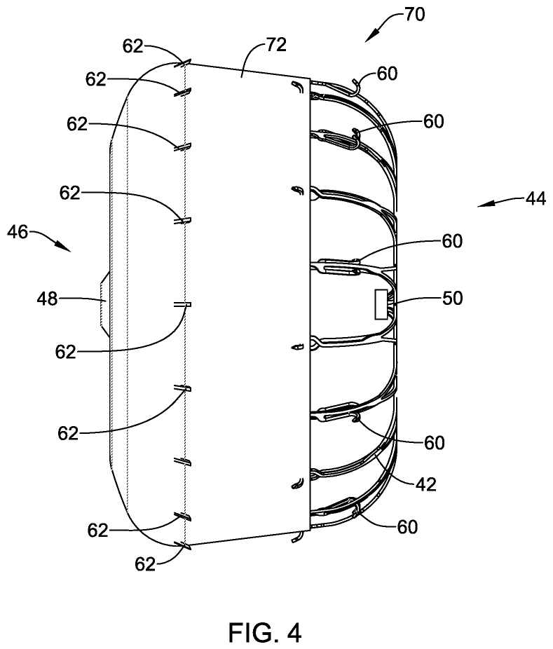

FIG. 4 is a side view of an illustrative LAAC device 70. The LAAC device 70 is similar to the LAAC device 40, but includes a membrane 72 extending over at least a portion of the expandable frame 42. As shown, the membrane 72 extends over the proximal region 46 of the expandable frame 42. The membrane 72 does not extend to where the proximally-facing fixation barbs 60 are located. The distally-facing fixation barbs 60 extend through the membrane 72 and thus the LAAC 70 is able to have bi-directional stability once successfully implanted. Further details regarding the construction of the LAAC device 40 (and the LAAC device 70) may be found in US Patent Application Publication No. 2017/0224354, which reference is incorporated by reference herein.

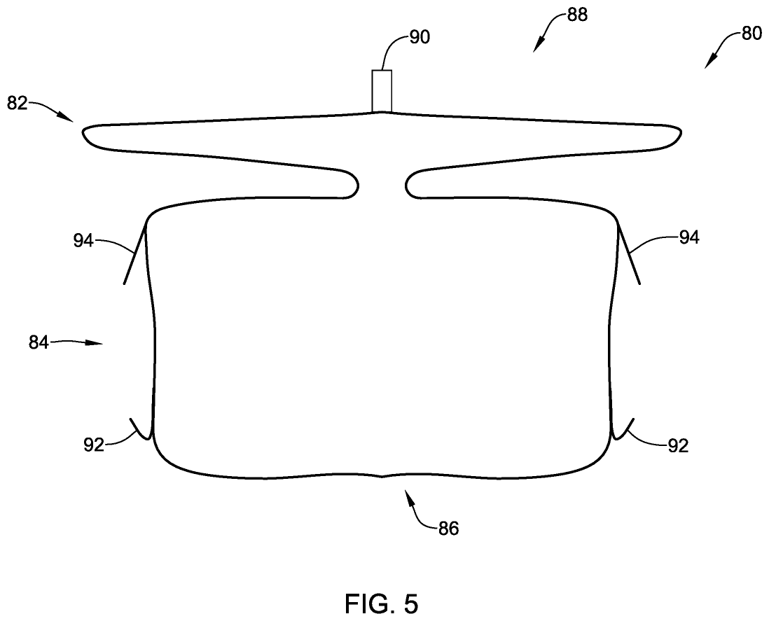

FIG. 5 is a schematic side view of an illustrative implantable medical device (IMD) 80. The IMD 80 includes a disk portion 82 and an expandable frame portion 84. For clarity, only an outline of the disk portion 82 and the expandable frame 84 is shown. It will be appreciated that the expandable frame 84 may include one or more, or even a plurality, of internal struts or other members that fill at least some of the space within the expandable frame 84 as shown.

The expandable frame 84 may be moveable between a collapsed configuration for delivery and an expanded configuration for deployment. In FIG. 5, the expandable frame 84 is shown in the expanded configuration. In some cases, the expandable frame 84 may be biased to the expanded configuration, and is adapted to permit collapse of the expandable frame 84 in response to a compressive force applied to the expandable frame 84. For example, pulling the expandable frame 84 into a delivery device may be construed as collapsing the expandable frame 84 into the collapsed configuration. Moving the expandable frame 84 out of the delivery device allows the expandable frame 84 to naturally regain the expanded configuration to which it is biased.

The IMD 80 may be considered as having a distal region 86 and a proximal region 88. In this, distal and proximal refer to the anticipated orientation of the IMD 80 once the IMD 80 has been successfully implanted. The IMD 80 includes a hub 90 that is disposed within the proximal region 88 of the IMD 80. The hub 90 may be used for releasably securing the IMD 80 to a delivery device (not shown), for example.

The IMD 80 includes one or more proximally-facing fixation barbs 92. While only two proximally-facing fixation barbs 92 are shown, it will be appreciated that the IMD 80 may include any number of proximally-facing fixation barbs 92, and may include three, four, five, six, seven, eight, nine, ten or more proximally-facing fixation barbs 92. The proximally-facing fixation barbs 92 may be integrally formed with the expandable frame 84, particularly if for example the expandable frame 84 is laser-cut. In some cases, the proximally-facing fixation barbs 92 may be formed of wire and subsequently secured to the expandable frame 84 using any desired technique such as but not limited to welding or soldering. The proximally-facing fixation barbs 92 may be considered as being proximally-facing when the IMD 80 has been implanted.

The IMD 80 includes one or more distally-facing fixation barbs 94. While only two distally-facing fixation barbs 94 are shown, it will be appreciated that the IMD 80 may include any number of distally-facing fixation barbs 94, and may include three, four, five, six, seven, eight, nine, ten or more distally-facing fixation barbs 94. The distally-facing fixation barbs 94 may be integrally formed with the expandable frame 84, particularly if for example the expandable frame 84 is laser-cut. In some cases, the distally-facing fixation barbs 94 may be formed of wire and subsequently secured to the expandable frame 84 using any desired technique such as but not limited to welding or soldering. The distally-facing fixation barbs 94 may be considered as being distally-facing when the IMD 10 has been implanted.

The proximally-facing fixation barbs 92 and the distally-facing fixation barbs 94 may be disposed anywhere on the expandable frame 84. In some instances, as shown, the proximally-facing fixation barbs 92 are located within the distal region 86 and the distally-facing fixation barbs 94 are located within the proximal region 88. In some cases, the proximally-facing fixation barbs 92 are located within the proximal region 88 and the distally-facing fixation barbs 94 are located within the distal region 86. In some instances, the proximally-facing fixation barbs 92 and the distally-facing fixation barbs 94 may be randomly distributed throughout the expandable frame 84.

While the proximally-facing fixation barbs 92 and the distally-facing fixation barbs 94 are shown and described as being either proximally-facing or distally-facing, it will be appreciated that in some instances, the fixation barbs may not necessarily be proximally-facing or distally-facing. In some cases, the fixation barbs may be considered to be divided into a first group of one or more fixation barbs that extend in a first direction, and a second group of one or more fixation barbs that extend in a second direction that oppose the first direction. The first group of fixation barbs may extend in a direction that is 180 degrees apart from the direction in which the second group of fixation barbs extend. The first group of fixation barbs may extend in a direction that is 170 degrees apart, or 160 degrees apart, or 150 degrees apart, or 140 degrees apart, or 130 degrees apart, or 120 degrees apart, or even less.

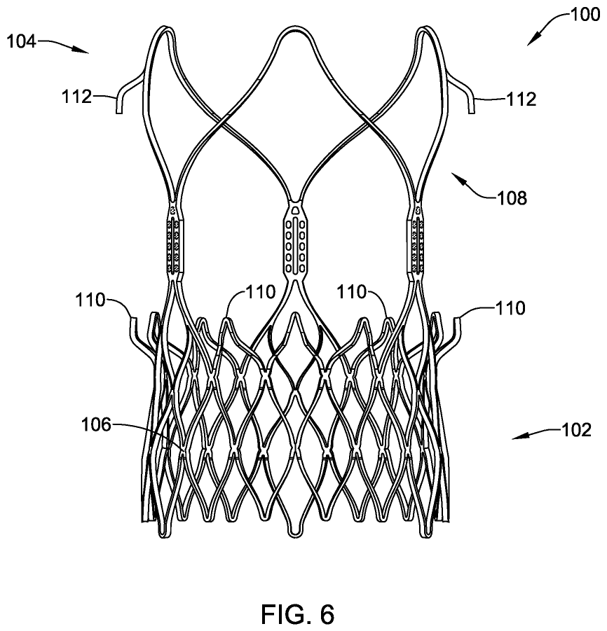

FIG. 6 is a side view of an illustrative aortic valve implant frame 100. For clarity, the rest of the aortic valve implant, such as the tissue forming the valve flaps, is not shown. The aortic valve implant frame 100 may be moveable between a collapsed configuration for delivery and an expanded configuration for deployment. In FIG. 6, the aortic valve implant frame 100 is shown in the expanded configuration. In some cases, the aortic valve implant frame 100 may be biased to the expanded configuration, and is adapted to permit collapse of the aortic valve implant frame 100 in response to a compressive force applied to the aortic valve implant frame 100. For example, pulling the aortic valve implant frame 100 into a delivery device may be construed as collapsing the aortic valve implant frame 100 into the collapsed configuration. Moving the aortic valve implant frame 100 out of the delivery device allows the aortic valve implant frame 100 to naturally regain the expanded configuration to which it is biased.

The aortic valve implant frame 100 may be considered as having a distal region 102 and a proximal region 104, since the aortic valve that includes the aortic valve implant frame 100 may be delivered to the aortic annulus via the aorta. The distal region 102 of the aortic valve implant frame 100 includes an annular portion 106 that is adapted to lodge within the aortic annulus, which can help to appropriately locate the replacement aortic valve. The proximal region 104 of the aortic valve implant frame 100 includes arched portions 108 that help to position and anchor the replacement aortic valve by interacting with the vessel walls within the aorta.

The distal region 102 includes a number of proximally-facing fixation barbs 110 that are formed as part of the annular portion 106. The proximal region 104 includes a number of distally-facing fixation barbs 112 that are formed as part of the arched portions 108. It will be appreciated that the distally-facing fixation barbs 112 are in opposition to the proximally-facing fixation barbs 110, meaning that movement in either axial direction will be constrained by either the distally-facing fixation barbs 112 or the proximally-facing fixation barbs 110. If the aortic valve implant frame 100 moves distally, meaning movement towards the native aortic valve, closer to the left ventricle, the distally-facing fixation barbs 112 will resist this motion. If the aortic valve implant frame 100 moves proximally, meaning movement away from the native aortic valve, closer to the aorta, the proximally-facing fixation barbs 110 will resist this motion.

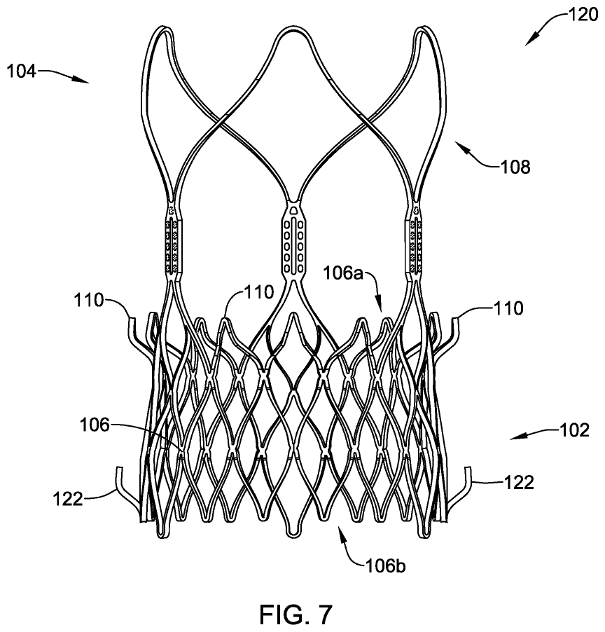

FIG. 7 is a side view of an illustrative aortic valve implant frame 120. For clarity, the rest of the aortic valve implant, such as the tissue forming the valve flaps, is not shown. The aortic valve implant frame 120 may be moveable between a collapsed configuration for delivery and an expanded configuration for deployment. In FIG. 7, the aortic valve implant frame 120 is shown in the expanded configuration. In some cases, the aortic valve implant frame 120 may be biased to the expanded configuration, and is adapted to permit collapse of the aortic valve implant frame 120 in response to a compressive force applied to the aortic valve implant frame 120. For example, pulling the aortic valve implant frame 120 into a delivery device may be construed as collapsing the aortic valve implant frame 120 into the collapsed configuration. Moving the aortic valve implant frame 120 out of the delivery device allows the aortic valve implant frame 100 to naturally regain the expanded configuration to which it is biased.

The aortic valve implant frame 120 may be considered as having a distal region 102 and a proximal region 104, since the aortic valve that includes the aortic valve implant frame 120 may be delivered to the aortic annulus via the aorta. The distal region 102 of the aortic valve implant frame 120 includes an annular portion 106 that is adapted to lodge within the aortic annulus, which can help to appropriately locate the replacement aortic valve. The proximal region 104 of the aortic valve implant frame 100 includes arched portions 108 that help to position and anchor the replacement aortic valve by interacting with the vessel walls within the aorta.

The distal region 102 includes a number of proximally-facing fixation barbs 110 that are formed as part of the annular portion 106 and are located at or near a proximal side 106a of the annular portion 106 as well as a number of proximally-facing barbs 122 that are formed as part of the annular portion 106 and are located at or near a distal side 106b of the annular portion 106. In some cases, the arched portion 108 may also include additional fixation barbs, such as the distally-facing fixation barbs 112 shown in FIG. 6.

The devices described herein, as well as various components thereof, may be manufactured according to essentially any suitable manufacturing technique including molding, casting, mechanical working, and the like, or any other suitable technique. Furthermore, the various structures may include materials commonly associated with medical devices such as metals, metal alloys, polymers, metal-polymer composites, ceramics, combinations thereof, and the like, or any other suitable material. These materials may include transparent or translucent materials to aid in visualization during the procedure. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; combinations thereof; and the like; or any other suitable material.

Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane, polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), polycarbonates, ionomers, biocompatible polymers, other suitable materials, or mixtures, combinations, copolymers thereof, polymer/metal composites, and the like.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device, comprising:
an expandable frame defining a body of the implantable medical device, the expandable frame moveable between a collapsed configuration for delivery and an expanded configuration for deployment;
one or more proximally-facing fixation burrs secured relative to the expandable frame; and
one or more distally-facing fixation burrs secured relative to the expandable frame;
wherein the one or more proximally-facing fixation burrs and the one or more distally-facing fixation burrs together are adapted to provide the implantable medical device with bi-directional stability.

2. The implantable medical device of claim 1, wherein the one or more proximally-facing fixation burrs are disposed within a distal region of the expandable frame.

3. The implantable medical device of claim 1, wherein the one or more distally-facing fixation burrs are disposed within a proximal region of the expandable frame.

4. The implantable medical device of claim 1, wherein the expandable frame comprises a plurality of struts.

5. The implantable medical device of claim 4, wherein the one or more distally-facing fixation burrs are secured to at least some of the plurality of struts.

6. The implantable medical device of claim 4, wherein the one or more proximally-facing fixation burrs are secured to at least some of the plurality of struts.

7. The implantable medical device of claim 1, wherein the implantable medical device comprises an LAAC (left atrial appendage closure) device.

8. The implantable medical device of claim 1, wherein the implantable medical device comprises a heart valve implant.

9. The implantable medical device of claim 1, wherein the expandable frame is biased to the expanded configuration.

10. A left atrial appendage closure device, comprising:
an expandable frame moveable between a collapsed configuration for delivery and an expanded configuration for deployment;
one or more proximally-facing fixation burrs secured relative to the expandable frame; and
one or more distally-facing fixation burrs secured relative to the expandable frame;
wherein the one or more proximally-facing fixation burrs and the one or more distally-facing fixation burrs together are adapted to provide the left atrial appendage closure device with bi-directional stability.

11. The left atrial appendage closure device of claim 10, wherein the one or more proximally-facing fixation burrs are disposed within a distal region of the expandable frame.

12. The left atrial appendage closure device of claim 10, wherein the one or more distally-facing fixation burrs are disposed within a proximal region of the expandable frame.

13. The left atrial appendage closure device of claim 10, wherein the expandable frame is adapted to enable the expandable frame to be partially recaptured within a delivery device for repositioning the expandable frame, wherein the distally-facing fixation burrs are disposed within the delivery device when the expandable frame is partially recaptured.

14. The left atrial appendage closure device of claim 10, wherein the expandable frame is biased to the expanded configuration.

15. The left atrial appendage closure device of claim 10, wherein the expandable frame comprises a shape memory material.

16. The left atrial appendage closure device of claim 10, wherein the expandable frame comprises a plurality of laser-cut struts, and the proximally-facing fixation struts are laser cut as part of the laser-cut struts.

17. The left atrial appendage closure device of claim 16, wherein the distally-facing fixation struts are welded to the laser-cut struts.

18. The left atrial appendage closure device of claim 10, further comprising a membrane covering at least a proximal portion of the expandable frame.

19. A left atrial appendage closure device, comprising:
an expandable braided frame moveable between a collapsed configuration for delivery and an expanded configuration for deployment;
one or more proximally-facing fixation burrs secured to the expandable braided frame; and
one or more distally-facing fixation burrs secured to the expandable braided frame;
wherein the one or more proximally-facing fixation burrs and the one or more distally-facing fixation burrs together are adapted to provide the left atrial appendage closure device with bi-directional stability.

20. The left atrial appendage closure device of claim 19, wherein the one or more proximally-facing fixation burrs and/or the one or more distally-facing fixation burrs comprise wire elements that are separately formed and subsequently attached to the expandable braided frame.
